(19) United States
(12) Patent Application Publication
(10) Pub. No.: US 2016/0120686 A1
(43) Pub. Date: May 5, 2016

(54) Orthopedic Back Support System

(71) Applicant: Eric S. Ritter, Weeki Wachee, FL (US)

(72) Inventor: Eric S. Ritter, Weeki Wachee, FL (US)

(21) Appl. No.: 14/995,935

(22) Filed: Jan. 14, 2016

Related U.S. Application Data
(63) Continuation of application No. 14/471,379, filed on Aug. 28, 2014, now abandoned.
Application No. 14/471,379 is a continuation of application No. 14/036,282, filed on Sep. 25, 2013, now abandoned.
Application No. 14/036,282 is a continuation of application No. 13/215,980, filed on Aug. 23, 2011, now abandoned.
(60) Provisional application No. 61/376,529, filed on Aug. 24, 2010.

(51) Int. Cl. A61F 5/02; A61F 5/30

(52) U.S. Cl. CPC A61F 5/028; A61F 5/30

(57) ABSTRACT

The present invention teaches a back support system for providing posture and orthopedic benefits to the user, comprising, in combination, a frontal support member, a rear support member, and at least one adjustable triple ply belting or strap formed to provide with an intermediary member a triple ply construction.

The application claims the benefit or priority pursuant to 35 U.S.C. 119(e) from a U.S. Provisional Patent Application having Application No. 61/376,529 filed Aug. 24, 2010.

### BACKGROUND OF THE INVENTION

The present invention relates generally providing support and bracing to the back of a human, and more particularly to aback support system with both posture and orthopedic benefits formed with novel pressure-inducing structural configurations.

There has long been a need for improved supports for the back in people who suffer from a myriad of ailments that would be helped with improved support. From the need for posture support to the needs of the injured and victims of degenerative types of ailments, a sturdy back support has been desired and needed.

There have been efforts to address these needs. An example can be found in the disclosure of a brace in U.S. Pat. No. 3,171,409. Another example can be found in the disclosure of an orthopedic support in U.S. Pat. No. 4,991,573. Still another example is found in a back support marketed by Lifeback Enterprises, Inc. of Tarpon Springs, Fla.

The present invention seeks to improve upon each and all of the prior art efforts to provide back support, by providing a novel structural configuration designed to add specific regional pressure to locations at the back of the user of the present invention. The present invention provides belt-like support in addition to its regional pressure point supporting configuration.

### SUMMARY OF THE INVENTION

The present invention contemplates the use of plastic as well as any number of desirable alternative materials whose structural properties will provide the supporting function necessary and/or desirable. This invention contemplates both unitary single member supports, as well as modular components which may be assembled, thereby providing smaller shipping container capabilities. My invention is suitable for males and females.

### BRIEF DESCRIPTION OF THE DRAWINGS

The present invention has been assigned an owner/operator number 10032237 by the U.S. Food and Drug Administration, covering an embodiment of the present invention.

The present invention provides a relatively low profile structural design which avoids what would otherwise cause limitations and restrictions of movement by the user. This enables a far more active and productive lifestyle for the user. Furthermore, the user is able to wear the present invention beneath clothing, such that the support is provided away from the view of others, Where desired, or over clothing where appearance is not adversely an issue.

Bilateral opposing forces provide pain relief and support to the user. Not only is support provided, but posture is improved and the present invention can be used to create posture-awareness in the wearer, thereby additional serving a posture-awareness educational and training function. Thus, pain is reduced while at the same time affording the user training in better posture for times during and after wearing the present invention.

The present invention provides lower lumbar orthotic-type support for people who have suffered lower back injuries or who suffer from skeletal degenerative diseases. By serving as an exoskeleton exterior support, the muscular and skeletal region of the lower back is assisted in a therapeutic fashion.

In particular, one or more elongated pressure pads of a predetermined geometric configuration are integrally formed in one or both of frontal and rear support members. These support members are joined with a triple ply belt, such that where said pressure pads are formed in both the frontal and rear support members, the user is able to enjoy substantially constant opposing therapeutic pressure between these pads.

The present invention contemplates a single pressure pad in each of the frontal and rear support members, as well as embodiments of the present invention wherein a plurality of such pressure pads are formed in either one or both of these support members.

The belt is adjustable by the user, to increase or decrease pressure between the pressure pads, for either comfort or therapeutic purposes. Superior Velcro-securing fastening means are provided.

While the foregoing has focused upon support for the back of the user of the present invention, it is important here to emphasize that abdominal support is simultaneously provided. Injuries to the spine are assisted neurologically, and core body muscles are permitted to function according to their intended functions, thereby avoiding atrophy and undesirable weakness. Where prescribed appropriately, the user of the present invention is able to prevent and protect against injury, in addition to facilitating recovery from injury that has already occurred.

One will appreciate that the delicate spinal system of discs and adjoining body parts are vulnerable to injuries caused during sports, work, irregular and incorrect movement, and normal stresses and strains. By providing support to the user of the present invention, pain is mitigated or prevented, not only in the lower back, but also in adjoining areas such as the legs. The invention induces better spinal alignment during all times when it is worn.

The present invention contemplates its being recommended by health professionals who will appreciate its structural therapeutic benefits, especially given the pressure pad configurations disclosed herein.

The above description, as well as further objects, features and advantages of the present invention will be more fully understood with reference to the following annexed informal drawings (formal drawings will accompany a non-provisional patent application deriving the benefit of the filing date of this provisional patent application), wherein:

FIG. 1 is a photograph of the system according to the present invention, in which the frontal and rear support members formed of plastic and of substantially uniform thickness are operationally joined in use by a triple ply adjustable belt;

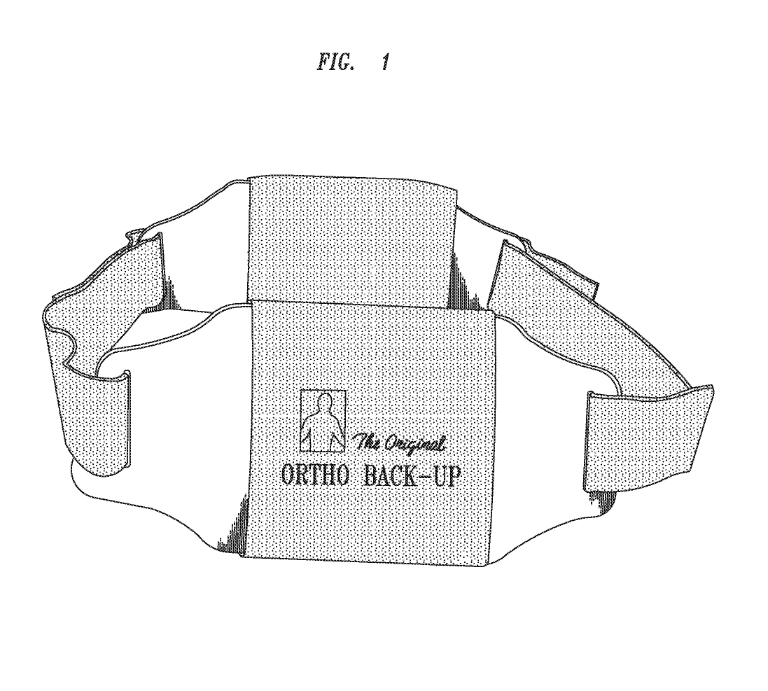

FIG. 2 is a front view of a support member according to an embodiment of the present invention formed with a single pressure pad of length L and height H;

FIG. 3 is a front view of a support member according to another embodiment of the present invention formed with a single pressure pad of length L and height H;

FIG. 4 is a front view of a support member according to yet another embodiment of the present invention formed with a plurality (in this instance, two) of pressure pads, of diameters D1 and D2, and spaced apart by a distance W; and

FIG. 5 is a pictorial sectional view through an adjustable strap or belting according to the present invention such as that shown in FIG. 1, illustrating a triple ply construction.

### DETAILED DESCRIPTION OF THE INVENTION

The present patent application focuses upon the nature and structural configurations of the frontal and rear members to be joined by the triple ply adjustable belt and more particularly draws the reader's attention to the various embodiments of the present invention wherein the dimensions and configurations of same may be varied.

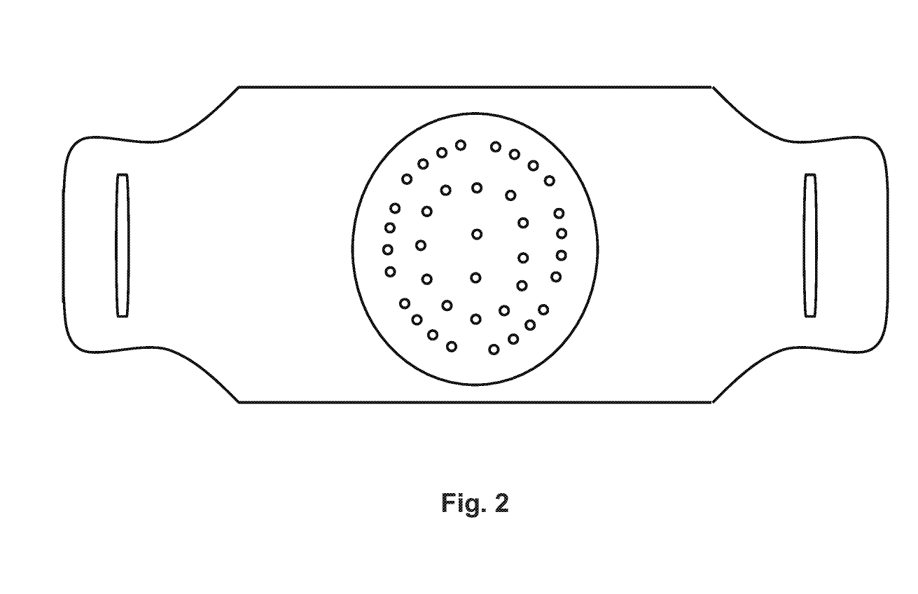

By way of example, in FIG. 2, the height H is greater in magnitude than the length L of the single pressure pad. This facilitates and enables a non-circular area of pressure to be applied to the back and/or abdominal area. Throughout the various embodiments of the present invention shown in the accompanying drawings, the layout, size and spacing of the ventilating openings may be varied, and may provide an aesthetic as well as functional purpose. It is contemplated by the present invention to provide such ventilating openings in the shape of a logo or design associated with the goodwill of the entity marketing the invention.

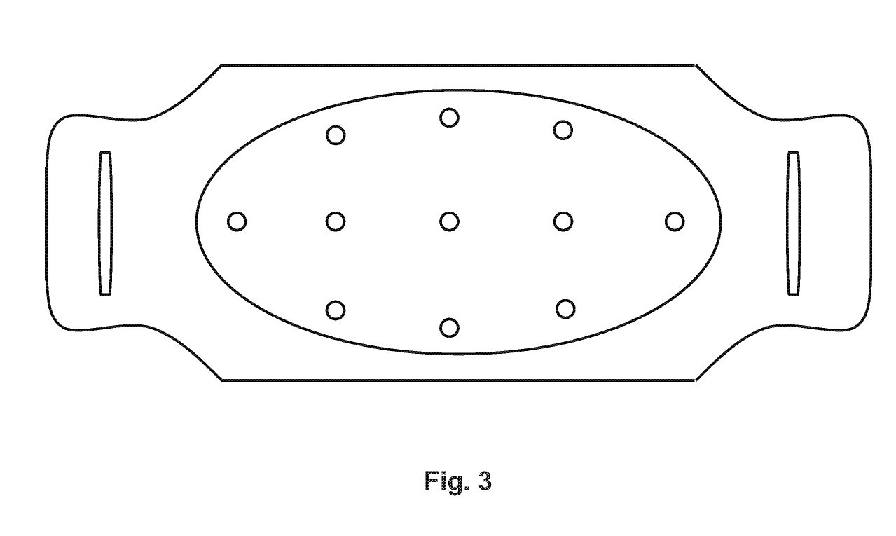

In FIG. 3, a greater elongated elliptical pressure pad of length L and height H is illustrated, where the ration of L/H is 6.5/11.5.

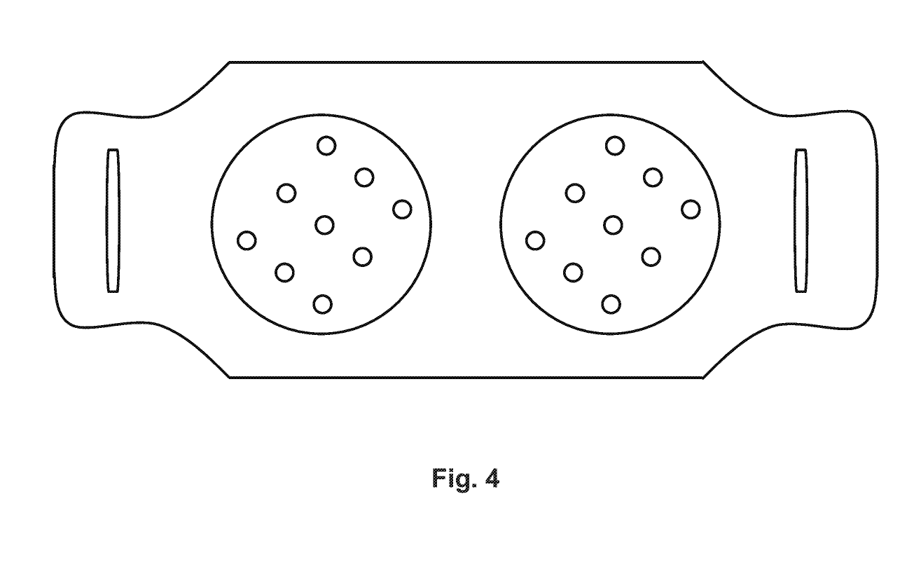

In FIG. 4, a pair of substantially circular pressure pads of diameters D1 and D2 are spaced apart from one another a distance W, to permit pressure upon the back of the user without putting pressure against the spine itself. It is contemplated that, where the user has injured the surface of the spinal lumbar area and where pressure on that surface will cause pain, the spaced-apart pressure pads will provide support without the pain associated with pressure directly upon the wounded spinal surface area. This will permit healing of such a wound during support, and will avoid irritation otherwise caused by movement of the wearer. Once the wound is healed, an alternative pressure pad according to the present invention can be substituted.

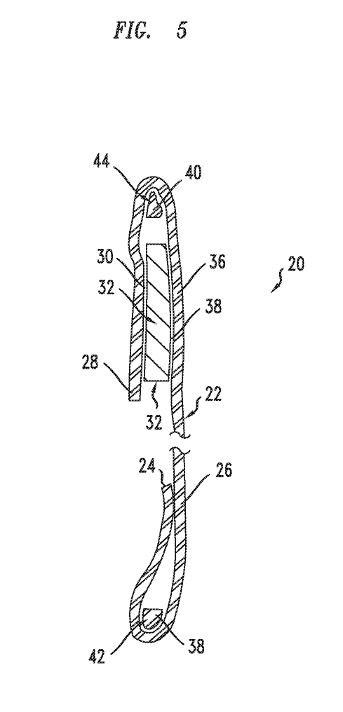

In FIG. 5, the adjustable strap or belting 20 includes an elongated first member 22 extending from a first end portion 24 thereof removably secured to a second portion 26 thereof a third end portion 28 thereof removably secured to one side 30 of an intermediary member 32, and a fourth portion 36 thereof spaced from said third end portion 28, removably secured to an opposite side 38 of said intermediary member 32, said belting 20 extending through portions 38 and 40 of said frontal and rear support members 42 and 44, said third end portion 28 and said intermediary member 32 and said fourth portion 36 comprising a triple ply construction in accordance to the present invention.

The present invention contemplates varying the dimensions of length L and height H such that, while they are not identical, they may accommodate a variety of user needs, depending upon their injuries or condition.

1. A back support system for providing posture and orthopedic benefits to the user, comprising, in combination,
a frontal support member formed with a plurality of air-penetrable ventilating openings therethrough,
a rear support member formed with a plurality of air-penetrable ventilation openings therethrough,
a protruding portion of said rear support member formed in an integral predetermined geometric pressure pad configuration, said geometric configuration being located at the intersection of vertical and horizontal reference axes, the length L of said geometric configuration along said horizontal reference ax is being of a different magnitude than the height H of the geometric configuration along the vertical reference axis, and
an adjustable triple ply belting cooperatively joining said frontal and rear support members.

2. The back support system of claim 1, wherein the L/H ratio of said length L to height W being substantially 6.0 to 7.0.

3. The back support system of claim 1, comprising a plurality of said protruding portions.

4. The back support system of claim 1, wherein the ration of length L to height H is greater than one (1).

5. The back support system of claim 1, wherein the ration of length L to height H is less than one (1).

6. The back support system of claim 1, wherein said belting includes an elongated first member extending from a first end portion thereof removably secured to a second portion thereof, a third end portion thereof removably secured to one side of an intermediary member, and a fourth portion thereof spaced from said third end portion, removably secured to an opposite side of said intermediary member, said belting extending through portions of said frontal and rear support members, said third end portion and said intermediary member and said fourth portion comprising a triple ply construction.
